(19) United States
(12) Patent Application Publication
(10) Pub. No.: US 2026/0174315 A1
(43) Pub. Date: Jun. 25, 2026

(54) ENDOSCOPE DEVICE AND SYSTEM WITH AN ENDOSCOPE DEVICE

(71) Applicant: KARL STORZ SE & Co. KG, Tuttlingen (DE)

(72) Inventor: Peter SCHWARZ, Tuttlingen (DE)

(21) Appl. No.: 19/422,775

(22) Filed: Dec. 17, 2025

(30) Foreign Application Priority Data
Dec. 19, 2024 (DE) 10 2024 138 868.4

(51) Int. Cl. A61B 1/00; A61B 1/05; A61B 1/313

(52) U.S. Cl. CPC A61B 1/00193; A61B 1/00103; A61B 1/00117; A61B 1/00119; A61B 1/05; A61B 1/3132

(57) ABSTRACT

The invention relates to an endoscope device (10), comprising: a shaft (20) with a shaft longitudinal axis (30); an actuating shaft (40) which is movable in parallel with the shaft longitudinal axis (30); and a camera module (50), which is pivotally mounted on the shaft (20) and has a stereoscopic image recording unit (60), wherein the camera module (50) can be deflected from an insertion position to an image capturing position by moving the actuating shaft (40) relative to the camera module (50) in parallel with the shaft longitudinal axis (30) and/or by moving the camera module (50) relative to the actuating shaft (40), wherein, in the image capturing position, the camera module (50) defines a viewing direction which is at an angle to the shaft longitudinal axis (30), and wherein, in the image capturing position, the stereoscopic image recording unit (60) is mounted so as to rotate relative to the actuating shaft (40) to rectify an image.

The invention further relates to a system with an endoscope device according to the invention.

The present invention relates to an endoscope device, in particular a stereoscopic laparoscope, and to a system with an endoscope device.

In minimally invasive surgery, endoscopes form part of the prior art, enabling the creation of magnified images of a region within a patient's body that is under examination. For improved visualization, stereoscopic image recording units are used in endoscopes and can visualize the environment to a surgeon and/or another user in 3-D and/or 2-D.

To give the surgeon and/or other user a good overview, the image recording units are usually angled toward a longitudinal axis of the endoscope in order to visualize images at an angle to the longitudinal axis of the endoscope and thus in a so-called oblique view. This usually involves the use of endoscopes that have a 30°, 45°, 60°, or 75° oblique view.

When designing endoscopes with a stereoscopic image recording unit that are intended to have an oblique view during use, it is especially difficult in particular to reduce the outer diameters in such a way that the minimally invasive procedure can be kept as small as possible. Furthermore, to improve user orientation in 3-D and/or 2-D representations, the image from the image recording unit is to be rectified, if necessary. While 2-D representations can also be rectified electronically, this is not possible with 3-D representations. Here, image rectification can be achieved, for example, through complex mechanical systems for moving the individual image sensors.

Furthermore, sufficient light must be provided at the distal end of the endoscope to adequately illuminate the inside of the patient's body in order to record the best possible images.

In addition, the medical imaging capability of endoscopes allows different types of tissue, such as organs and/or blood vessels and/or tumor tissue, to be visualized at different depths under the skin or in the body cavity. Fluorescence imaging methods are used to be able to better differentiate the tissue types. For this purpose, the patient is given a drug containing a fluorescent dye, in particular fluorophores, which is deposited in one of the different types of tissue. Particularly with these fluorescence imaging methods, sufficient excitation light must be provided at the distal end of the endoscope in order to be able to adequately excite the fluorescent substances.

In known endoscopes with an oblique view, distal prisms are used to achieve the oblique view. Especially with oblique-view endoscopes, the integration of sufficient illumination fibers presents a significant challenge, since the distal prisms severely restrict the space within the endoscope for integrating illumination fibers.

Furthermore, it is important to be able to flush the distal end of the endoscope during use. A fluid is usually used to flush the distal end of the endoscope and is guided to the distal end of the endoscope via corresponding lines. However, integrating the lines into known endoscopes sometimes requires an additional flushing shaft, which must also be attached to the endoscope. The additional shaft unnecessarily increases the outer diameter of the endoscope. Therefore, due to the additional shaft, a trocar must be used, which has a larger diameter and unnecessarily enlarges the minimally invasive procedure.

DE10004264C2 discloses a stereo camera that can be spread apart from the shaft of an endoscope via joints. By advancing an instrument, the spreading is controlled, and the instrument is kept within the camera's field of view.

WO2014104405A1 describes an endoscope with a stereo camera that can be positioned at different angles and can be moved into the angled position by a thrust mechanism.

A similar mechanism is also known from EP2123225A1.

US11064867B2 discloses an endoscope wherein a distal camera unit is deflected outward by advancing a hollow shaft within the instrument and is thus moved into a position that is laterally offset relative to the shaft.

Proceeding from the prior art, the object addressed by the invention is in particular that of creating an endoscope with an oblique view that enables a user to intuitively rectify an image, but is not limited thereto.

Furthermore, proceeding from the prior art, the object addressed by the invention is in particular that of providing with the smallest possible minimally invasive procedure and plenty of space, e.g., for the integration of illumination fibers and/or fluid lines, but is not limited thereto.

At least one of these objects is achieved according to the invention by an endoscope device and by a system with an endoscope device, as are described herein and defined in the claims.

The present invention provides an endoscope device with a shaft having a shaft longitudinal axis. The endoscope device further comprises an actuating shaft that is movable in parallel with the shaft longitudinal axis. Furthermore, the endoscope device comprises a camera module that is pivotally mounted on the shaft. The camera module also comprises a stereoscopic image recording unit. The camera module can be deflected from an insertion position to an image capturing position by moving the actuating shaft in parallel with the shaft longitudinal axis relative to the camera module, in particular into a position next to the camera module. Alternatively and/or additionally, the camera module can be deflected from an insertion position to an image capturing position by moving the camera module relative to the actuating shaft, in particular into a position next to the actuating shaft. In the image capturing position, the camera module defines a viewing direction that is at an angle to the shaft longitudinal axis. Additionally, in the image capturing position, the stereoscopic image recording unit can be mechanically rotated to rectify an image. In particular, in the image capturing position, the stereoscopic image recording unit can be mechanically rotated relative to the actuating shaft to rectify an image. For example, in the image capturing position, the stereoscopic image recording unit is mounted so as to rotate relative to the actuating shaft to rectify an image.

In particular, the endoscope device is inserted inside the patient's body in the insertion position. For example, the endoscope device can be inserted inside the body through a trocar. In the image capturing position, the endoscope device in particular has an oblique view. The oblique view can, for example, be at an angle of 30° or 45° or 60° or 75°. For example, in the image capturing position, the endoscope device can be designed to record an image of an environment, such as the inside of the patient's body.

In particular, the shaft extends from a proximal end to a distal end of the endoscope device. In particular, the shaft is rigid and/or flexible. In particular, the shaft is round and/or angular. Preferably, the shaft is made entirely or partially of metal, plastic, and/or ceramic. The longitudinal axis of the shaft extends in particular from the proximal end to the distal end of the shaft. In particular, the longitudinal axis of the shaft passes through a center point of a cross-section of the shaft and/or in parallel with a main direction of extension of the shaft.

The camera module in particular surrounds the stereoscopic image recording unit. The image recording unit is in particular received in the camera module and thus protected by the camera module and/or by a housing of the camera module.

In particular, the stereoscopic camera module is arranged at the distal end of the actuating shaft in the insertion position. The stereoscopic camera module is in particular movably fastened to the shaft or pivotally mounted on the shaft. The actuating shaft is attached in particular to the shaft. The stereoscopic camera module comprises a stereoscopic image recording unit.

The stereoscopic image recording unit can comprise a first image recording apparatus and a second image recording apparatus, each of which is sensitive to light within a spectral range. Furthermore, the stereoscopic image recording unit can comprise a third image recording apparatus and/or a fourth image recording apparatus, which, for example, are sensitive to light within a further spectral range. In particular, the stereoscopic image recording unit is a 3-D camera system. The first image recording apparatus and/or the second image recording apparatus may, in particular, comprise sensors and prisms mounted back-to-back. Furthermore, the first image recording apparatus and/or the second image recording apparatus and/or the third image recording apparatus and/or the fourth image recording apparatus may comprise filters and/or an optical system.

Using the first image recording apparatus and the second image recording apparatus, for example, stereoscopic images can be recorded within a first spectral range. Using the third image recording apparatus and/or the fourth image recording apparatus, for example, stereoscopic images can be recorded within a second spectral range. This allows fluorescence stereoscopic images to be recorded using the third image recording apparatus and/or the fourth image recording apparatus. To enable fluorescence imaging, the image recording apparatuses can comprise additional image sensors that are sensitive to light within different spectral ranges. The first image recording apparatus and the second image recording apparatus can each comprise a first image sensor that is sensitive to light at least predominantly within the first spectral range, which is particularly associated with visible light. This means that it is possible to record images within the visible light wavelength range using the first image sensor. This corresponds, for example, to white light image recording. The third image recording apparatus and the fourth image recording apparatus can each comprise a second image sensor that is sensitive to light at least predominantly within the second spectral range, which is particularly associated with near-infrared light. This means that it is possible to record images within the near-infrared light wavelength range using the second image sensor.

Alternatively, the first and second image recording apparatuses can be sensitive in both the visual and near-infrared ranges and record images, such as fluorescence images, in both wavelength ranges.

The stereoscopic camera module is deflected from the insertion position to the image capturing position by moving the actuating shaft, in particular by advancing the actuating shaft toward a distal end of the actuating shaft. Alternatively and/or additionally, the camera module can be deflected from the insertion position to the image capturing position by retracting the camera module toward a proximal end of the camera module. In other words, the actuating shaft can be moved in parallel with the shaft longitudinal axis next to the camera module, in particular, by advancing the actuating shaft toward the inside of the patient's body next to the camera module and/or by moving the camera module back out from inside the patient's body next to the actuating shaft. The oblique view of the stereoscopic image recording unit is achieved by deflecting the stereoscopic camera module. In other words, by deflecting the stereoscopic camera module, the camera module defines a viewing direction that is at an angle to the longitudinal axis of the shaft. The oblique view can, for example, be at an angle of 30°, 45°, 60°, and/or 75°.

In particular, the stereoscopic camera module and/or the stereoscopic image recording unit comprise(s) an input optical system that defines an optical axis. The viewing direction extends in particular along the optical axis.

In the image capturing position, the stereoscopic image recording unit can rotate relative to the camera module and/or relative to the shaft and/or relative to the actuating shaft. In particular, the stereoscopic image recording unit is mechanically rotatable for rectifying an image. For example, the stereoscopic image recording unit is rotatably mounted for this purpose.

Image rectification is used to rectify an image that is recorded, for example, by the camera module. When the endoscope device is rotated by a user, the image recording unit is also rotated, causing the image to be rotated on a screen. Such a rotational movement can make orientation difficult for the user, since the spatial directions within the image and in reality differ. Thus, an “up” direction, e.g., relative to the horizon or the direction of gravity, may be rotated downward or laterally in the image. To improve orientation, it is common practice to electronically counter-rotate normal images. However, this is not possible with stereoscopic images, since a rotational movement of the endoscopic device also means a rotational movement of the stereo base. This cannot be counter-rotated electronically. It is advantageously possible in this case to also rectify a stereoscopic image by rotating the image recording unit so that a stereoscopic impression is maintained, and the spatial directions in the image correspond to reality or to a user setting.

In particular, the stereoscopic image recording unit is also rotatably mounted in the insertion position of the camera module (50). The stereoscopic image recording unit can be rotatable in a manner that is not dependent upon the image capturing position. This makes it possible to decouple the image rectification process of the stereoscopic image recording unit from the image capturing position and/or the oblique view of the endoscope.

To ensure easy or smooth rotation of the image recording unit, the image recording unit can be rotatably mounted within the camera module. For example, the stereoscopic image recording unit can, however, also be designed to rotate together with the camera module. In this case, the camera module can be mounted on the actuating shaft, in particular in the image capturing configuration.

For ease of use, the image recording unit can be rotated using a cable, in particular to rectify an image. The cable can, in particular, be a torsion cable and/or a power supply cable. The torsion cable can, for example, be routed directly along the power supply cable and/or reinforce the power supply cable and/or encase the power supply cable. The cable can be connected to an electronics unit, in particular a camera control unit (CCU), or the cable can connect the camera module to the electronics unit. The rotational movement of the cable can be achieved, for example, by a transmission, which is electrically driven, for example. The cable can also be rotated manually-for example, by the user. Alternatively and/or additionally, the cable can also be rotated together with the electronics unit.

To enable the endoscope device to be inserted inside the body so as to save as much space as possible, the cable can be received in the shaft in the insertion position. This makes it possible to use trocars with the smallest possible diameter in order to keep the minimally invasive procedure as small as possible. Furthermore, the shaft may, for example, have a slot in which the cable can be received. In the image capturing position, the cable may in particular protrude from the shaft. As a result, the cable can be rotated very smoothly, and the image rectification process can therefore be adjusted very smoothly.

In the image capturing position, the actuating shaft can at least partially receive the stereoscopic camera module. In particular, the actuating shaft receives the stereoscopic camera module, to provide the oblique view of the stereoscopic image recording unit. Furthermore, in particular to provide the oblique view in the image capturing position, a surface normal of a distal end surface of the actuating shaft can be angled or at an angle to the shaft longitudinal axis. For example, the surface normal of the distal end surface of the actuating shaft runs in parallel with the optical axis of the camera module.

For example, to reduce contamination between the camera module and the actuating shaft, a distal end of the actuating shaft can be flush with a distal end of the camera module, especially in the image capturing position. “Flush” means, in particular, that minor deviations between the distal end of the actuating shaft and the distal end of the camera module are permitted.

The actuating shaft may, in particular, have a receptacle in which the camera module is at least partially received. The camera module can be pressed onto the receptacle or be biased against it, especially in the image capturing position. The receptacle may also be at an angle, in particular with respect to a longitudinal axis of the actuating shaft and/or the shaft longitudinal axis. Furthermore, the receptacle can be adapted to the shape of the camera module. For example, the receptacle may form and/or have a semicircular and/or angular and/or U-shaped depression. Additionally, the camera module can be mounted so as to rotate relative to the receptacle.

To ensure that the camera module rests securely on the actuating shaft and/or the receptacle, the camera module can rest on the actuating shaft, in particular on the receptacle of the actuating shaft, especially in the image capturing position. Furthermore, especially in the image capturing position, the camera module can be biased against the actuating shaft, in particular against the receptacle of the actuating shaft, and in particular pressed against it.

To define the movement between the camera module and the shaft, the camera module can be pivotally mounted on the shaft by means of a pivoting device.

In order to securely position the camera module, the camera module can be pressed against the actuating shaft, in particular against the receptacle of the actuating shaft, by the pivoting device.

To define the movement between the camera module and the shaft and to ensure smooth pivoting, the pivoting device has a first hinge joint and a second hinge joint. The first hinge joint and the second hinge joint can be spaced apart from one another. In particular, the first hinge joint is connected to the shaft, and the second hinge joint is connected to the camera module.

To ensure that the camera module is securely positioned, the first hinge joint and/or the second hinge joint can have a torsion spring that presses the camera module onto the actuating shaft in the image capturing position.

In order to be able to illuminate the inside of the body, the actuating shaft can have at least one light-decoupling surface designed to decouple illumination light toward the distal end. The light-decoupling surface can be formed in particular by illumination fibers and/or optical systems and/or LED's.

The actuating shaft may comprise at least one optical fiber and/or illumination fibers, and/or at least one fluid line, which extend along a longitudinal axis of the actuating shaft. Since the fluid lines extend along the longitudinal axis of the actuating shaft, they are in particular not bent and therefore have a higher degree of durability. Furthermore, this results in particular in less friction loss of the fluid and allows for an at least largely laminar flow of the fluid.

Additionally, the actuating shaft may have at least one opening for an optical fiber in order to decouple illumination light toward the distal end. Alternatively and/or additionally, the actuating shaft may have at least one opening for a fluid line in order to be able to clean the distal end of the actuating shaft, in particular by means of a fluid, in particular air, in particular compressed air, and/or gas, in particular sterile gas, and/or water. In particular, the fluid cleans the camera module, in particular the image recording unit, and/or the light-decoupling surface and/or the at least one opening of the optical fiber and/or at least one opening of the fluid lines. Alternatively and/or additionally, a fluid, in particular compressed air, and/or gas, in particular sterile gas, and/or water and/or blood and/or other bodily fluids, can furthermore be aspirated from the distal end through the at least one fluid line. In particular, the optical fiber and/or the fluid line can exit the actuating shaft through at least one opening. In particular, the illumination light from the optical fiber and/or the fluid from the fluid line can exit the actuating shaft through the opening, and/or the fluid can be aspirated from the distal end through the opening.

For cost-effective production, the actuating shaft in particular is made of plastic. Furthermore, the at least one fluid line of the actuating shaft can be connected to at least one supply line. By connecting the at least one supply line to the at least one fluid line of the actuating shaft, the supply lines can be designed to be flexible at a proximal end of the at least one supply line. This allows the at least one supply line to be connected directly to a pump unit and/or insufflation unit without a separate interface.

Furthermore, at least one component of the actuating shaft and/or the actuating shaft can be designed as a disposable part in order to be able to reduce cleaning costs.

To ensure that virtually no fluid escapes from the endoscope device at an undesirable point, the actuating shaft can be sealed with respect to the shaft by means of a sealing element, in particular by means of an O-ring.

In order to be able to better adapt the actuating shaft to the functions it fulfills, or to be able to better adapt individual parts of the actuating shaft to their functions and/or use, the actuating shaft may in particular have a receiving shaft. In particular, the receiving shaft has a channel into which the shaft can be inserted.

In order to also be able to better adapt the actuating shaft to each of the functions it fulfills, or to be able to better adapt individual parts of the actuating shaft to their functions and/or use, the actuating shaft may, in particular, comprise an illumination rod in addition to or as an alternative to the receiving shaft. In particular, the illumination rod may have at least one light-decoupling surface, in particular light-decoupling illumination fibers and/or a light-decoupling optical system, designed to decouple illumination light toward the distal end.

To achieve a simple modular design of the actuating shaft, the receiving shaft has a channel into which the illumination rod can be inserted.

To provide illumination light, the illumination rod can comprise at least one optical fiber extending along a longitudinal axis of the illumination rod. For example, the optical fiber can be coupled to the light-decoupling optical element.

For cost-effective manufacture, the receiving shaft can be made of plastic. In particular, the receiving shaft can be designed as a disposable part so that it does not re-quire complex cleaning.

To extend the service life, at least a distal end of the illumination rod or part of the illumination rod can be made of metal. In particular, the illumination rod is designed to be reusable and, in particular, autoclavable.

In order to be able to flexibly connect the optical fiber to a light source, the optical fiber can, outside the patient's body or outside the trocar at a proximal end of the illumination rod and/or the actuating shaft, continue to be guided to the light source in a flexible tube. In particular, the optical fiber can run continuously from the distal end to the proximal end of the illumination rod and/or the actuating shaft. For example, the optical fiber runs from the distal end to the proximal end of the illumination rod and/or the actuating shaft without interruption and/or without an additional interface. By avoiding the interface or by ensuring that the optical fiber runs without interruption and/or continuously from the proximal end to the distal end of the illumination rod and/or the actuating shaft, loss of light can be greatly reduced. In this connection, the inventors recognized that the loss of light can be reduced by 30% compared to similar optical fibers that are connected to one another via an interface.

To increase the lifespan and light output of the optical fiber, the optical fiber can also be pre-bent and/or polished at a distal end of the illumination rod and/or the actuating shaft in the viewing direction. This means that it may in particular be provided that, for each viewing direction, a different illumination rod and/or actuating shaft be provided, which is flexibly interchangeable. In particular, the various interchangeable illumination rods and/or actuating shafts can be used flexibly with a shaft that stays the same, on which the camera module is pivotally mounted. Furthermore, the optical fiber may be glued into the illumination rod and/or the actuating shaft, in particular at the distal end of the illumination rod and/or the actuating shaft.

To prevent the fluid from escaping between the shaft and the receiving shaft, the receiving shaft may have at least one sealing element, in particular an O-ring, which creates a seal with respect to the shaft. To prevent the fluid from escaping between the illumination rod and the receiving shaft, the receiving shaft can have at least one sealing element, in particular an O-ring, which creates a seal with respect to the illumination rod.

To achieve a simple modular design, the receiving shaft in particular can have the receptacle in which the camera module is at least partially received. To define the camera module, especially when capturing an image, the camera module can be pressed in particular onto the receptacle.

To achieve the oblique view of the camera module and/or the image recording unit, the receptacle can be at an angle to a longitudinal axis of the actuating shaft and/or to a longitudinal axis of the receiving shaft. For example, the receptacle can be designed as a semi-circular and/or angular and/or U-shaped depression to be able to securely receive the camera module. Alternatively or additionally, the receptacle can be adapted to the shape of the image recording unit.

To ensure simple and/or user-friendly image rectification, the camera module in particular can be mounted so as to rotate with respect to the receptacle.

To define the position of the camera when capturing an image, the camera module can rest on the receiving shaft, especially in the image capturing position. In particular, to achieve the oblique view of the endoscope device, a surface normal of a distal end surface of the receiving shaft and/or the illumination rod can run at an angle to the longitudinal axis of the shaft. In particular, in the image capturing position, a distal end of the receiving shaft and/or the illumination rod can additionally or alternatively be flush with a distal end of the camera module for this purpose.

In particular, the receiving shaft may comprise at least one fluid line extending along a longitudinal axis of the receiving shaft. This prevents the fluid line from being bent in the receiving shaft. This increases the service life of the fluid line and reduces the friction losses of the fluid that is conveyed through the fluid line. The receiving shaft may, in particular, have at least one opening for a fluid line. For example, the fluid line and/or the fluid can exit the receiving shaft through at least one opening.

In addition, the invention comprises a system with an endoscope device, as it is described herein and defined in the claims, and a further actuating shaft which can be used instead of the actuating shaft. To achieve multiple viewing directions and/or oblique views, the actuating shaft can, in particular, define a first image capturing position having a first viewing direction. Furthermore, the additional actuating shaft can in particular define a second image capturing position having a second viewing direction that is different from the first viewing direction. Therefore, the system can use a plurality of actuating shafts to realize different viewing directions and/or oblique views, each providing a different oblique view and/or a different viewing direction.

Furthermore, the invention comprises a system with an endoscope device, as it is described herein and defined in the claims, comprising an actuating shaft having a receiving shaft and an illumination rod. The actuating rod in particular comprises at least one further receiving shaft which can be used in place of the receiving shaft. To achieve multiple viewing directions and/or oblique views, the receiving shaft can in particular define a first image capturing position having a first viewing direction. Furthermore, the additional receiving shaft can in particular define a second image capturing position having a second viewing direction that is different from the first viewing direction. Therefore, the system can use a plurality of different receiving shafts to achieve different viewing directions and/or oblique views, each providing a different oblique view and/or a different viewing direction. The illumination rod, on the other hand, can be designed in such a way that it can be used with the different receiving shafts. Alternatively, however, an additional illumination rod can also be used for each receiving shaft and in particular has a light-emitting surface that shines in the viewing direction realized by the particular receiving shaft.

The present invention will be described by way of example below with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using numerical words.

In the drawings:

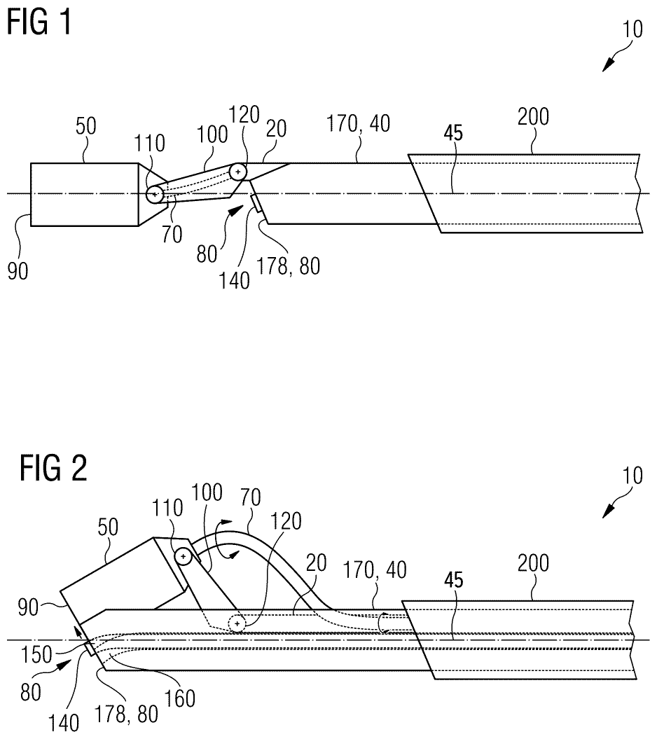

FIG. 1 shows an exemplary embodiment of an endoscope device according to the invention;

FIG. 2 shows an exemplary embodiment of an endoscope device according to the invention in an image capturing position;

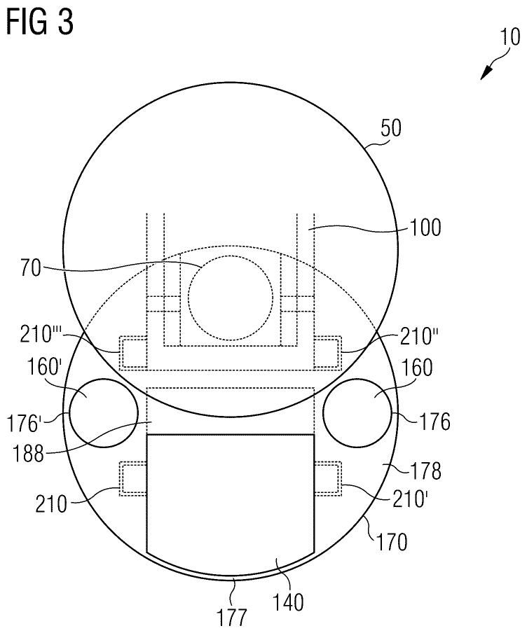

FIG. 3 shows a distal view of an exemplary embodiment of an endoscope device according to the invention in the image capturing position;

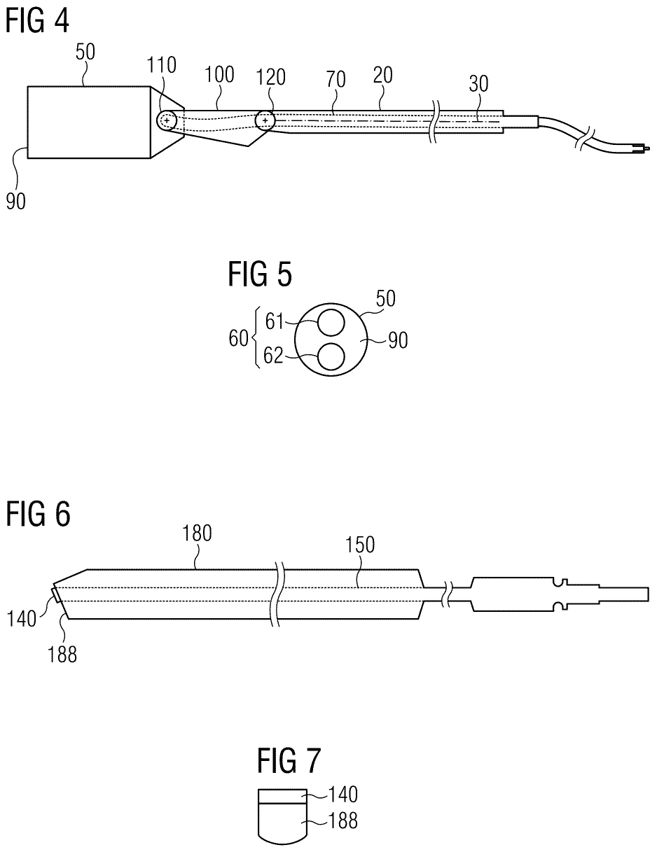

FIG. 4 shows an exemplary embodiment of a stereoscopic camera module according to the invention, which is pivotally mounted on the shaft;

FIG. 5 shows a distal view of the exemplary embodiment shown in FIG. 4;

FIG. 6 shows an exemplary embodiment of an illumination rod according to the invention;

FIG. 7 shows a distal view of the exemplary embodiment shown in FIG. 6;

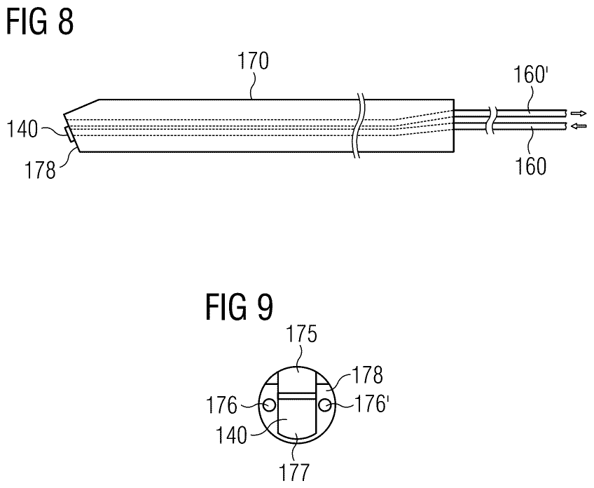

FIG. 8 shows an exemplary embodiment of a receiving shaft according to the invention; and

FIG. 9 shows a distal view of an exemplary embodiment of a receiving shaft according to the invention.

FIG. 1 shows an exemplary embodiment of an endoscope device 10 according to the invention in an insertion position in which the endoscope device 10 can be inserted into the inside of a patient's body-for example, by means of a trocar 200. FIG. 2 shows an exemplary embodiment of an endoscope device 10 according to the invention in an image capturing position of the camera module 50.

The endoscope device 10 comprises, in both embodiments shown in FIG. 1 and FIG. 2, a stereoscopic camera module 50, which is pivotally arranged on a shaft 20, and an actuating shaft 40. The shaft 20 has a shaft longitudinal axis 30 that extends from a proximal end to a distal end of the shaft 20. For example, the shaft 20 can pass through the actuating shaft 40. For example, the camera module 50 can be pivotally arranged on the shaft 20 by means of a pivoting device 100. In particular, the pivoting device 100 comprises a first hinge joint 110 and a second hinge joint 120 as well as a connecting piece connecting the first hinge joint 110 and the second hinge joint 120 to one another. The first hinge joint 110 can, for example, be connected to the camera module 50, and the second hinge joint 120 can, for example, be connected to the shaft 20.

The second hinge joint 120 can, for example, be mounted on one side of the shaft 20 and/or on both sides of the shaft 20. The second hinge joint 120 can, for example, comprise a spindle that passes through the shaft 20 and is rigidly connected to the connecting piece. The second hinge joint 120 can therefore be mounted so as to rotate relative to the shaft 20 by means of the spindle. In particular, said hinge joint can be mounted by means of a sliding bearing between the shaft 20 and the spindle.

The first hinge joint 110 can, for example, be mounted on one side of the camera module 50 and/or on both sides of the camera module 50. The first hinge joint 110 can, for example, comprise a spindle that passes through the camera module 50 and is rigidly connected to the connecting piece. The first hinge joint 110 can therefore be mounted so as to rotate relative to the camera module 50 by means of the spindle. In particular, said hinge joint can be mounted by means of a sliding bearing between the camera module 50 and the spindle.

Additionally, the camera module 50 can be connected to a cable 70. The camera module can be rotated in the image capturing position, in particular to rectify an image, via the cable 70. In particular, the cable 70 can comprise a power supply cable and a torsion cable. For example, the torsion cable can encase the power supply cable In order for the pivoting device 100 to press the camera module 50 onto the actuating shaft 40, the first hinge joint 110 and/or the second hinge joint 120 may have a torsion spring. The torsion spring can be biased by deflecting the pivoting device 100 by moving the actuating shaft 40 next to the camera module 50. In particular, the torsion spring is biased in such a way that the torsion spring exerts a rotational moment on the pivoting device 100. The rotational moment allows the camera module 50 to be pressed onto the actuating shaft 40.

The actuating shaft 40 and/or the receiving shaft 170 described later can, for example, have a receptacle 175 in which the camera module 50 can be received, in order to define the image capturing position of the camera module 50. In particular, the receptacle 175 can have a round and/or angular geometry. Preferably, the receptacle 175 is adapted to the shape of the camera module 50.

An optical fiber 150 can pass through the actuating shaft 40, the light from which exits the actuating shaft 40 via a light-decoupling surface 140 and which extends along a longitudinal axis 45 of the actuating shaft 40. The light-decoupling surface 140 is, for example, arranged at the distal end of the actuating shaft 40. The light-decoupling surface 140 can, alternatively and/or additionally, be formed by, for example, an LED. Preferably, the light-decoupling surface 140 is at an angle to the shaft longitudinal axis 30 similar and/or identical to the angle between the camera module 50 and the shaft longitudinal axis 30 in the image capturing position. That is, the light-decoupling surface 140 is preferably oriented in a direction that corresponds to the oblique view of the endoscope device 10 in the image capturing position.

Furthermore, at least one fluid line 160 can be passed through the actuating shaft 40 and extend along the longitudinal axis 45 of the actuating shaft 40. Through the at least one fluid line 160, a fluid, e.g., water and/or air and/or gas, can be guided from a proximal end of the actuating shaft 40 to a distal end of the actuating shaft 40.

In particular, the fluid exits from the actuating shaft 40 via at least one opening. The at least one opening is arranged in particular at the distal end of the actuating shaft 40. For example and/or preferably, the distal end of the actuating shaft 40 is at an angle to the shaft longitudinal axis 30 similar and/or identical to the angle between the camera module 50 and the shaft longitudinal axis 30 in the image capturing position. That is, the at least one opening is preferably oriented in a direction that corresponds to the oblique view of the endoscope device 10 in the image capturing position.

The actuating shaft 40 can comprise a receiving shaft 170 and an illumination rod 180. The actuating shaft 40 can also be designed as a receiving shaft 170. The receiving shaft 170 and the illumination rod 180 can be separate components. In particular, the illumination rod 180 can pass through the receiving shaft 170, or the illumination rod 180 can be inserted through the receiving shaft 170. The receiving shaft 170 and the illumination rod 180 can, however, also form one component or a combined component. Furthermore, the receiving shaft 170 can have a cavity for receiving the shaft 20.

The receiving shaft 170 can be designed to receive the camera module 50 in the image capturing position and thus realize the oblique view of the camera module 50.

Furthermore, the receiving shaft 170 can comprise the at least one fluid line 160 and the at least one opening for the at least one fluid line. For example, the receiving shaft 170 is made of plastic. Furthermore, the receiving shaft 170 may be produced in such a way that it is suitable only for single use.

The illumination rod 180 can comprise the at least one optical fiber 150. The optical fiber 150 can run from a proximal end of the illumination rod 180 to a distal end of the illumination rod 180 and exit from the distal end 178 of the receiving shaft 180 in the form of the light-decoupling surface 140. For example, the illumination rod 180 is at least partially formed from, and/or encased by, a durable material, such as metal. As shown in FIG. 1, the camera module 50 can be arranged at a distal end of the actuating shaft 40, for the insertion of the endoscope device 10 through the trocar. This allows the endoscope device 10 to be inserted inside the patient's body through the trocar 200, even though the camera module 50 and the actuating shaft 40 both occupy an inner diameter of the trocar.

As shown in FIG. 2, by moving and/or advancing the actuating shaft 40 toward the inside of the patient next to the camera module 50 and/or by moving the camera module 50 back out from inside the patient next to the actuating shaft 40, the camera module 50 can be deflected by the pivoting device 100 in such a way that the camera module 50 is pressed onto the actuating shaft 40 in the image capturing position. In particular, in the image capturing position, a distal end 90 of the camera module 50 is flush with a distal end 80 of the actuating shaft 40. For example, the distal end 90 of the camera module 50 can, in the image capturing position, run in particular in parallel with the distal end 80 of the actuating shaft 40. he camera module 50 is mounted so as to be rotatable relative to the actuating shaft 40, as indicated by an arrow. The camera module 50 can be rotated manually or by a motor, e.g., by rotating the cable 70 connected to the camera module 50, to rotate the stereo base of the camera module and rectify an image.

FIG. 3 shows a distal view of an exemplary embodiment of an endoscope device 10 according to the invention in the image capturing position. FIG. 3 shows the distal end 90 of the camera module 50 and the distal end 80 of the actuating shaft 40. In the exemplary embodiment shown in FIG. 3, the actuating shaft 40 comprises the receiving shaft 170 and the illumination rod 180. Therefore, FIG. 3 also shows the distal end 178 of the receiving shaft 170 and the distal end 188 of the illumination rod 180. The camera module 50 is connected to the shaft on the proximal side by means of the pivoting device 100. Furthermore, the camera module is connected to the cable 70 on the proximal side. In the distal view of the exemplary embodiment of the endoscope device 10 shown in FIG. 3, the pivoting device 100 and the cable 70 are arranged behind the camera module 50 and are therefore hidden by the camera module 50. Therefore, the pivoting device 100 and the cable 70 are shown by dashed lines.

The illumination rod 180 can in particular be inserted into the receiving shaft 170. To ensure that the illumination rod 180 can be occupied as completely as possible by the optical fiber and/or at least one optical fiber, the illumination rod 180 can have guides, in particular guide lugs, 210, 210′, 210″, 210″ on its edge, by means of which the illumination rod 180 can be fixed in the receiving shaft 170. A light-decoupling surface 140 is arranged at the distal end 188 of the illumination rod 180.

The distal end 178 of the receiving shaft 170 has openings 176 and 176′for the fluid lines 160 and 160′. In particular, the openings 176 and 176′are located between the guides 210, 210′, 210″, 210′″. Preferably, the openings 176 and 176′are arranged such that they can clean the camera module 50, in particular an image recording unit (not shown) of the camera module 50, and/or the light-decoupling surface 140.

Furthermore, the receiving shaft 170 has a receptacle 175 by which the camera module 150 is received in the image capturing position. Preferably, the shape of the receptacle 175 is adapted to the shape of the camera module 150. For example, the receptacle 175 can have a semi-circular shape, and the camera module 150 can have a round shape.

The receptacle 175 can be at an angle to the longitudinal axis 45 of the actuating shaft 40 and/or to the longitudinal axis of the receiving shaft 170.

FIG. 4 shows an exemplary embodiment of the stereoscopic camera module 50 according to the invention, which is pivotally mounted on the shaft 20 by means of the pivoting device 100. The pivoting device 100 can comprise a first hinge joint 110 and a second hinge joint 120. In particular, the first hinge joint 110 is connected to the camera module 50, and the second hinge joint 120 is connected to the shaft 20.

The shaft 20 has a shaft longitudinal axis 30. The camera module 50 is connected to the cable 70. The cable 70 can be guided through the shaft 20, as shown in FIG. 4.

The shaft 20 can have a slot or, for example, a U-shaped receptacle into which the cable 70 is inserted. That is, in the insertion position, the cable 70 can be received into the shaft 20, and, in the image capturing position, the cable 70 can protrude from the shaft 20 or the cable 70 can be driven out of the shaft 20 by the deflection of the camera module 50 into the image capturing position. This allows the camera module 50 to be rotated smoothly in the image capturing position relative to the actuating shaft 40 and the receptacle 175 by means of the cable 70. However, it is also possible that the cable 70 is guided through the shaft 20 in both the insertion position and the image capturing position. For example, the cable 70 can also be routed through pivoting device 100. This can reduce the risk of injury at undesired points inside the patient's body.

FIG. 5 shows a distal view of the exemplary embodiment shown in FIG. 4, or, rather, the distal end 90 of the camera module 50. A stereoscopic image recording unit 60 is integrated into the camera module 50. The image recording unit 60 comprises a first image recording apparatus 61 and a second image recording apparatus 62. The first image recording apparatus 61 and the second image recording apparatus 62 may be spaced apart at a stereo base distance.

FIG. 6 shows an exemplary embodiment of the illumination rod 180 according to the invention. At the distal end 188, the illumination rod 180 has a light-decoupling sur-face 140. For example, the light-decoupling surface 140 can, as shown, protrude from the illumination rod 180 at the distal end 188. However, it is also possible that the light-decoupling surface 140 is integrated into the illumination rod 180 in such a way that the light-decoupling surface 140 does not protrude from the illumination rod 180. The light-decoupling surface 140 is connected to an optical fiber 150, which passes through the illumination rod 180. In particular, the optical fiber 150 has no interruptions and/or interfaces, such that the amount of light transported through the optical fiber 150 to the light-decoupling surface 140 can be increased. The distal end 188 of the illumination rod 180 is at an angle in particular to the vertical of the shaft longitudinal axis and/or to the vertical of a longitudinal axis of the illumination rod 180. In particular, the distal end 188 of the illumination rod 180 is at such an angle that the distal end 188 is flush with the camera module 50 in the image capturing position. In other words, the distal end 188 of the illumination rod 180 is at such an angle that the distal end 188 runs perpendicularly and/or approximately perpendicularly to the viewing direction of the camera module 50 in the image capturing position.

FIG. 7 shows a distal view of the exemplary embodiment shown in FIG. 6, or, rather, the distal end 188 of the illumination rod 180 with the light-decoupling surface 140.

FIG. 8 shows an exemplary embodiment of the receiving shaft 170 according to the invention, in which the illumination rod 180 is received. The light-decoupling surface 140 of the illumination rod 180 protrudes from the receiving shaft 170. Furthermore, the receiving shaft 170 has a fluid line 160 and a fluid line 160′which pass through the receiving shaft 170. For example, a fluid, in particular air and/or water and/or sterile gas, can be guided to the distal end 178 of the receiving shaft 170 via the fluid line 160. In addition, for example, another fluid, such as a contaminated fluid, in particular blood and/or air and/or water and/or sterile gas, can be aspirated from the distal end 178 of the receiving shaft 170 via the fluid line 160′.

FIG. 9 shows a distal view of an exemplary embodiment shown in FIG. 8, or, rather, the distal end 178 of the receiving shaft 170. The distal end 178 of the receiving shaft 170 has an opening 176 for the fluid line 160 and an opening 176′for the fluid line 160′. Additionally, the distal end 178 of the receiving shaft 170 can have an opening 177 for the light-decoupling surface 140. For example, the opening 176 of the fluid line 160 and the opening 176′ of the fluid line 160′ can be arranged on opposite sides of the opening 177 for the light-decoupling surface 140. In particular, the opening 176 of the fluid line 160 and the opening 176′of the fluid line 160′ can be arranged at the same height on opposite sides of the opening 177 for the light-decoupling surface 140. Furthermore, the distal end 178 of the receiving shaft 170 can have the receptacle 175 in which the camera module 50 can be received.

### LIST OF REFERENCE SIGNS

10 endoscope device
20 shaft
30 longitudinal shaft axis
40 actuating shaft
45 longitudinal axis
50 camera module
60 image recording unit
61 first image recording apparatus
62 second image recording apparatus
70 cable
80 distal end of the actuating shaft
90 distal end of the camera module
100 pivoting device
110 first hinge joint
120 second hinge joint
140 light-decoupling surface
150 optical fiber
160 fluid line
170 receiving shaft
175 receptacle
176 opening for the fluid line
177 opening for the light-decoupling surface
178 distal end of the receiving shaft
180 illumination rod
188 distal end of the illumination rod
200 trocar
210, 210′, 210″, 210″′ guide

1. An endoscope device comprising:
a shaft having a shaft longitudinal axis;
an actuating shaft configured to move in parallel with the shaft longitudinal axis; and
a camera module, which is pivotally mounted on the shaft and has a stereoscopic image recording unit,
wherein the camera module is configured to be deflected from an insertion position to an image capturing position by moving the actuating shaft relative to the camera module and/or by moving the camera module relative to the actuating shaft, this movement being parallel to the shaft longitudinal axis,
wherein, in the image capturing position, the camera module defines a viewing direction which is at an angle to the shaft longitudinal axis and
wherein, in the image capturing position, the stereoscopic image recording unit is mounted so-as-to rotate relative to the actuating shaft to rectify an image.

2. The endoscope device according to claim 1,
wherein the image recording unit is also rotatably mounted when the camera module is in the insertion position.

3. The endoscope device according to claim 1, wherein the image recording unit is rotatably mounted within the camera module.

4. The endoscope device according to claim 1,
wherein the image recording unit is rotatable by one or more of a cable, a torsion cable and a power supply cable, to rectify the image.

5. The endoscope device according to claim 1,
wherein, in the image capturing position, the actuating shaft at least partially receives the camera module.

6. The endoscope device according to claim 1,
wherein, in the image capturing position, a surface normal of a distal end surface of the actuating shaft is at an angle to the shaft longitudinal axis.

7. The endoscope device according to claim 1,
wherein, in the image capturing position, a distal end of the actuating shaft is flush with a distal end of the camera module

8. The endoscope device according to claim 1,
wherein, in the image capturing position, the camera module rests on the actuating shaft, and in the camera module is biased against the actuating shaft

9. The endoscope device according to claim 1,
wherein the camera module is pivotally mounted on the shaft by means a pivoting device.

10. The endoscope device according to claim 9,
wherein the camera module is pressed against the actuating shaft by the pivoting device.

11. The endoscope device according to claim 9,
wherein the pivoting device has a first hinge joint and a second hinge joint which are spaced apart from one another,
wherein the first hinge joint is connected to the shaft, and wherein the second hinge joint is connected to the camera module.

12. The endoscope device according to claim 11,
wherein the first hinge joint and/or the second hinge joint has/have a torsion spring by which the camera module is pressed onto the actuating shaft in the image capturing position.

13. The endoscope device according to claim 1,
wherein the actuating shaft has at least one light-decoupling surface
configured to decouple illumination light toward the distal end.

14. The endoscope device according to claim 1,
wherein the actuating shaft comprises at least one optical fiber and/or at least one fluid line which extend along a longitudinal axis of the actuating shaft.

15. The endoscope device according to claim 1,
wherein the actuating shaft has at least one opening for an optical fiber and/or a fluid line.

16. The endoscope device according to claim 1,
wherein the actuating shaft is made of plastic.

17. The endoscope device according to claim 1,
wherein at least one component of the actuating shaft and/or the actuating shaft is/are a disposable part.

18. The endoscope device according to claim 1,
wherein the actuating shaft is sealed with respect to the shaft by a sealing element.

19. The endoscope device according to claim 1,
wherein the actuating shaft comprises a receiving shaft wherein the receiving shaft has a channel into which the shaft is inserted.

20. The endoscope device according to claim 1,
wherein the actuating shaft comprises an illumination rod which has at least one light-decoupling surface configured to decouple illumination light toward the distal end.

21. The endoscope device according to claim 19,
wherein the receiving shaft has a channel into which the illumination rod is inserted.

22. The endoscope device according to claim 21,
wherein the illumination rod comprises at least one optical fiber extending along a longitudinal axis of the illumination rod.

23. The endoscope device according to claim 19,
wherein the receiving shaft is a disposable part; and/or
wherein the illumination rod is reusable and autoclavable.

24. The endoscope device according to claim 19,
wherein the receiving shaft has at least one sealing element, which creates a seal with respect to the shaft, and/or
wherein the receiving shaft has at least one sealing element, which creates a seal with respect to the illumination rod.

25. The endoscope device according to claim 1,
wherein the actuating shaft and/or the receiving shaft has/have a receptacle in which the camera module is at least partially received.

26. The endoscope device according to claim 25,
wherein the camera module is pressed onto the receptacle.

27. The endoscope device according to claim 25,
wherein the receptacle is at an angle to a longitudinal axis of the actuating shaft and/or to a longitudinal axis of the receiving shaft.

28. The endoscope device according to claim 25,
wherein the receptacle is designed as a semicircular and/or angular depression.

29. The endoscope device according to claim 25,
wherein the receptacle is adapted to the shape of the camera module.

30. The endoscope device according to claim 25,
wherein the camera module is mounted so as to be rotatable with respect to the receptacle.

31. A system, comprising:
an endoscope device according to claim 1; and
another actuating shaft which can be used in place of the actuating shaft,
wherein the actuating shaft defines a first image capturing position having a first viewing direction and wherein the further actuating shaft defines a second image capturing position having a second viewing direction that differs from the first viewing direction.
